(19) United States
(12) Patent Application Publication
(10) Pub. No.: US 2022/0065809 A1
(43) Pub. Date: Mar. 3, 2022

(54) GAS SENSOR

(71) Applicant: DENSO CORPORATION, Kariya-city (JP)

(72) Inventors: Shota HAGINO, Nisshin-city (JP); Satoshi NAKAMURA, Nisshin-city (JP); Masato OZAWA, Kariya-city (JP)

(21) Appl. No.: 17/522,343

(22) Filed: Nov. 9, 2021

Related U.S. Application Data
(63) Continuation of application No. PCT/JP2020/016506, filed on Apr. 15, 2020.

(30) Foreign Application Priority Data
May 10, 2019 (JP) 2019-089953

(51) Int. Cl. G01N 27/407; G01N 33/00; G01N 27/406

(52) U.S. Cl. CPC G01N 27/4078; G01N 33/0009; G01N 27/409; G01N 27/4077; G01N 27/4067

(57) ABSTRACT

A gas sensor includes a sensor element, a housing, a contact terminal, an insulator, a lead wire, a sealing member, and a base-side cover. A circular protrusion of the housing is provided with a root portion connected to a flange and a diameter reduction portion which is located on the base side of the root portion in an axial direction and whose outer periphery has a diameter reduced with respect to that of the root portion. An outer diameter of the diameter reduction portion is smaller than an outer diameter of the root portion. An end portion on the tip side of the base-side cover in the axial direction is jointed to the outer periphery of the diameter reduction portion.

### CROSS-REFERENCE TO RELATED APPLICATION

The present application is based on and claims the benefit of priority from earlier Japanese Patent Application No. 2019-089953 filed on May 10, 2019, the description of which is incorporated herein by reference.

### BACKGROUND

### Technical Field

The present disclosure relates to a gas sensor including a sensor element.

### Related Art

For example, an in-vehicle gas sensor is disposed in an exhaust pipe of an internal-combustion engine of a vehicle, and is used to obtain an air-fuel ratio of the internal-combustion engine based on a gas to be detected, which is an exhaust gas flowing through the exhaust pipe, a concentration of oxygen in the gas to be detected, or the like.

### SUMMARY

As an aspect of the present disclosure, a gas sensor is provided which includes:

a sensor element having a detector capable of detecting gas and located on a tip side in an axial direction;

a cylindrical housing into which the sensor element is inserted in a state in which the detector protrudes to a tip side in the axial direction;

a contact terminal that contacts a terminal portion provided on a base side position of the sensor element in the axial direction;

an insulator that holds the contact terminal;

a lead wire that is connected to the contact terminal and is externally drawn out;

a sealing member that holds the lead wire; and

a base-side cover that is attached to an outer periphery of a circular protrusion provided to protrude from a flange, which configures a maximum outer diameter portion of the housing, to a base side in the axial direction, and holds the sealing member on an inner periphery side.

The circular protrusion is provided with a root portion that protrudes to the base side of the flange in the axial direction, a diameter reduction portion which is located on the base side of the root portion in the axial direction and whose outer periphery has a diameter reduced with respect to that of the root portion to form a circular step between the diameter reduction portion and the root portion, and a general portion which is located on the base side of the diameter reduction portion in the axial direction and whose outer periphery has a diameter reduced with respect to that of the diameter reduction portion to form a circular step between the general portion and the diameter reduction portion.

A tip-side end portion of the base-side cover in the axial direction is jointed to the outer periphery of the diameter reduction portion.

A circular gap whose width in a radial direction orthogonal to the axial direction is smaller than a thickness of the base-side cover is provided between the base-side cover and the general portion.

### BRIEF DESCRIPTION OF THE DRAWINGS

In the accompanying drawings:

FIG. 1 is a drawing illustrating a cross section of a gas sensor according to a first embodiment;

FIG. 2 is a drawing illustrating a cross section cut along the line II-II of FIG. 1 according to the first embodiment;

FIG. 3 is a drawing illustrating an enlarged cross section of a circular protrusion of a housing and the periphery of an end portion of a base-side cover on a tip-side in an axial direction according to the first embodiment;

FIG. 4 is a drawing illustrating a further enlarged main part shown in FIG. 3 according to the first embodiment;

FIG. 5 is a drawing illustrating an enlarged cross section of a sensor element of the gas sensor according to the first embodiment;

FIG. 6 is a drawing illustrating a cross section cut along the line VI-VI of FIG. 5 according to the first embodiment;

FIG. 7 is a drawing illustrating a cross section cut along the line VII-VII of FIG. 5 according to the first embodiment;

FIG. 8 is a drawing illustrating a cross section of another gas sensor according to the first embodiment;

FIG. 9 is a drawing illustrating an enlarged cross section of a circular protrusion of a housing and the periphery of an end portion of a base-side cover on a tip-side in an axial direction according to a second embodiment;

FIG. 10 is a drawing illustrating a further enlarged main part shown in FIG. 9 according to the second embodiment;

FIG. 11 is a drawing illustrating an enlarged cross section of a circular protrusion of a housing and the periphery of an end portion of a base-side cover on a tip-side in an axial direction according to a third embodiment;

FIG. 12 is a drawing illustrating a further enlarged main part shown in FIG. 11 according to the third embodiment;

FIG. 13 is a drawing illustrating an enlarged cross section of a circular protrusion of a housing and the periphery of an end portion of a base-side cover on a tip-side in an axial direction according to a fourth embodiment;

FIG. 14 is a drawing illustrating a further enlarged main part shown in FIG. 13 according to the fourth embodiment; and

FIG. 15 is a drawing illustrating an enlarged cross section of a base-side portion of a gas sensor in an axial direction according to a fifth embodiment.

### DETAILED DESCRIPTION OF THE PREFERRED EMBODIMENTS

For example, an in-vehicle gas sensor is disposed in an exhaust pipe of an internal-combustion engine of a vehicle, and is used to obtain an air-fuel ratio of the internal-combustion engine based on a gas to be detected, which is an exhaust gas flowing through the exhaust pipe, a concentration of oxygen in the gas to be detected, or the like. In the gas sensor, a sensor element having a detector is inserted into a cylindrical housing, the detector of the sensor element is covered with a tip-side cover attached to the housing, and a wiring part of the sensor element is covered with a base-side cover attached to the housing. The detector of the sensor element and the tip-side cover are disposed in the exhaust pipe, and the base-side cover is disposed outside the exhaust pipe.

When fuel in the internal-combustion engine burns, an exhaust gas passing through the exhaust pipe of the internal-combustion engine reaches a high temperature, for example, 1100° C. Then, heat is transferred from the exhaust pipe heated to high temperature to the housing fitted to the exhaust pipe, and then the heat is transferred from the housing to the base-side cover. In addition, a rubber sealing member for holding a lead wire electrically connected to the sensor element is disposed to the inner periphery side the base-side cover. Hence, the heat is also transferred from the base-side cover to the sealing member.

As a technique for protecting a sealing member from heat, a gas sensor is disclosed in, for example, JP 2015-99110 A. In the gas sensor, a thermal insulation space serving as an atmospheric layer having high heat insulating properties is provided between a sensor element and a grommet (sealing member), to prevent the grommet from deteriorating by heat.

In the gas sensor of JP 2015-99110 A, since an area in which a base-side cover contacts a housing is large, heat transfer from the housing to the base-side cover cannot be sufficiently suppressed. Diligent studies by the inventors found the necessity to further devise a structure of a contact portion between the housing and the base-side cover to suppress the heat transfer from the housing to the base-side cover.

The present disclosure provides a gas sensor that makes it difficult to heat a sealing member by heat transferred from a housing, whereby the sealing member can be effectively protected from the heat.

Preferred embodiments of a gas sensor will be described with reference to the drawings.

### First Embodiment

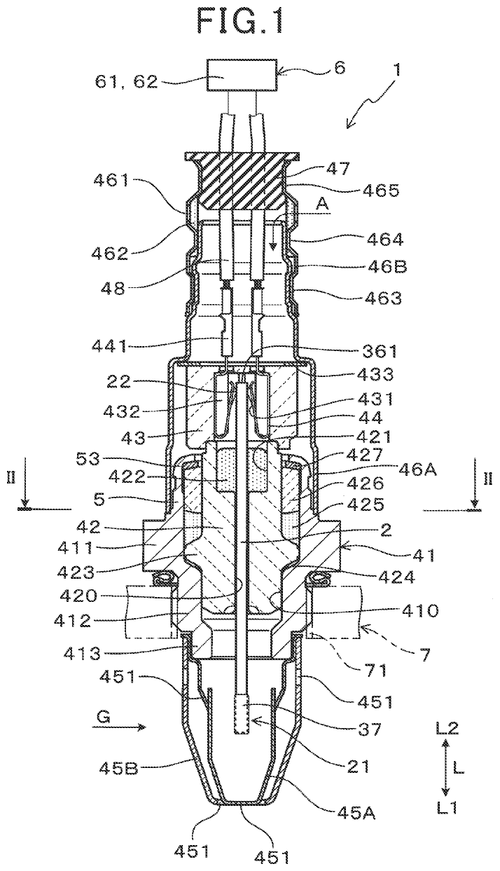

As shown in FIG. 1, a gas sensor 1 of the present embodiment includes a sensor element 2, a housing 41, a contact terminal 44, a second insulator 43, a lead wire 48, a sealing member 47, and base-side covers 46A, 46B. The sensor element 2 has a detector 21, which can detect gas, located on the tip side L1 in an axial direction L. The housing 41 has a cylindrical shape. The sensor element 2 is inserted to the inner periphery side of the housing 41. The detector 21 of the sensor element 2 protrudes from an end face of the housing 41 located on the tip side L1 in an axial direction L.

The contact terminal 44 contacts a terminal portion 22 provided on the base side L2 of the sensor element 2 in the axial direction L. The second insulator 43 holds the contact terminal 44. The lead wire 48 is connected to the contact terminal 44 and is externally drawn out from the gas sensor 1. The sealing member 47 holds the lead wire 48. The base-side cover 46A is attached to the outer periphery of a circular protrusion 5 provided so as to protrude from a flange 411, which configures a maximum outer diameter portion of the housing 41, toward the base side L2 in the axial direction L, and holds the sealing member 47 on the inner periphery side of the base-side cover 46A.

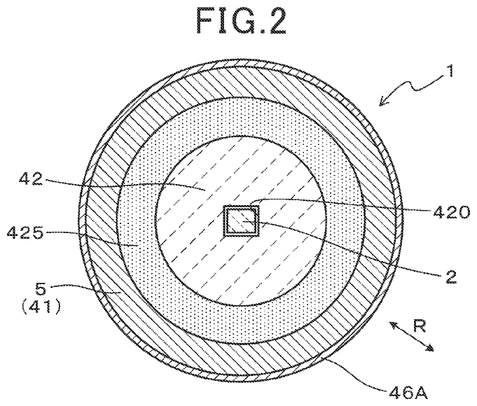

As shown in FIGS. 2 to 4, the circular protrusion 5 has a root portion 51 connected to the flange 411, and a diameter reduction portion 52 which is located on the base side L2 of the root portion 51 in the axial direction L and whose outer periphery has a diameter reduced with respect to that of the root portion 51. The outer diameter of the diameter reduction portion 52 is smaller than the outer diameter of the root portion 51. An end portion 460 on the tip side L1 of the base-side cover 46A in the axial direction L is jointed to the outer periphery of the diameter reduction portion 52.

Hereinafter, the gas sensor 1 of the present embodiment will be described in detail.

### (Gas Sensor 1)

As shown in FIG. 1, the gas sensor 1 is disposed to a mounting opening 71 of an exhaust pipe 7 of an internal-combustion engine of a vehicle, and is used to detect a concentration of oxygen or the like in a gas to be detected, which is an exhaust gas G flowing through the exhaust pipe 7. The gas sensor 1 can be used as an air-fuel ratio sensor (A/F sensor) that obtains an air-fuel ratio in the internal-combustion engine based on a concentration of oxygen, a concentration of unburned gas, and the like in the exhaust gas G. The air-fuel ratio sensor can quantitatively and continuously detect an air-fuel ratio from a fuel rich state in which a ratio of fuel to air is higher than a theoretical air fuel ratio to a fuel lean state in which the ratio of fuel to air is lower than the theoretical air fuel ratio. The gas sensor 1 can be used for, in addition to an air-fuel ratio sensor, various applications for obtaining a concentration of oxygen.

In the exhaust pipe 7, a catalyst is disposed to purify harmful substances in the exhaust gas G. The gas sensor 1 can be disposed either upstream or downstream of the catalyst in the direction in which the exhaust gas G flows in the exhaust pipe 7. The gas sensor 1 can also be disposed on the intake side of a pipe of a supercharger that increases a density of air sucked by the internal-combustion engine by using the exhaust gas G. The pipe in which the gas sensor 1 is disposed may be a pipe of an exhaust gas recirculation mechanism that recirculates part of the exhaust gas G, which is exhausted from the internal-combustion engine to the exhaust pipe 7, to an intake pipe of the internal-combustion engine.

### (Gas Sensor 2)

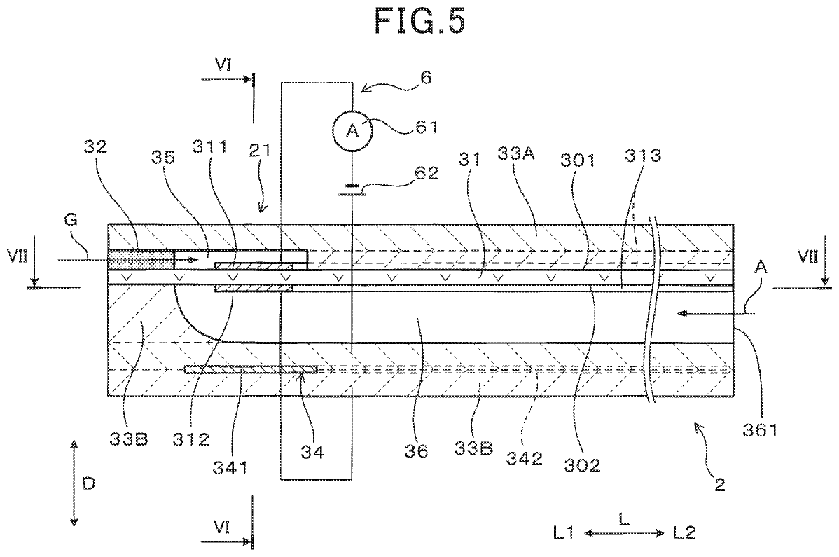

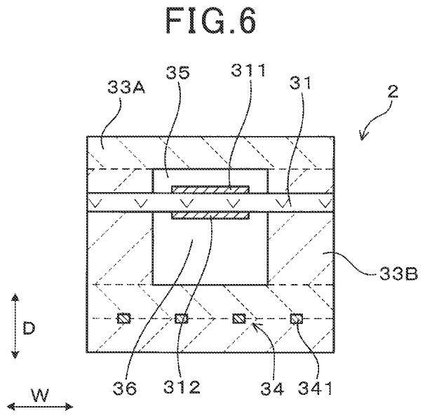

As shown in FIG. 5 and FIG. 6, a sensor element 2 of the present embodiment has a long rectangular shape, and includes a solid electrolyte element 31, an exhaust electrode 311, an air electrode 312, a first insulator 33A, a second insulator 33B, a gas chamber 35, an air duct 36, and a heating element 34. The sensor element 2 is a lamination type configured by laminating the insulators 33A, 33B and the heating element 34 to the solid electrolyte element 31.

In the present embodiment, the axial direction L of the sensor element 2 is a direction in which the sensor element 2 extends in enlarged shape. The direction which is orthogonal to the axial direction L and in which the solid electrolyte element 31 and the insulators 33A and 33B are laminated, in other words, the direction in which the solid electrolyte element 31, the insulators 33A and 33B, and the heating element 34 are laminated is referred to as a lamination direction D. The direction orthogonal to the axial direction L and the lamination direction D is referred to as a width direction W. The side (end) of the sensor element 2 in the axial direction L which is exposed to the exhaust gas G is referred to as a tip side L1, and the side (end) opposite to the tip side L1 is referred to as a base side L2.

### (Solid Electrolyte Element 31, Exhaust Electrode 311, Air Electrode 312)

As shown in FIG. 5 and FIG. 6, the solid electrolyte element 31 has conductivity of oxygen ions (O²) at a predetermined activation temperature. A first surface 301 of the solid electrolyte element 31 is provided with the exhaust electrode 311 exposed to the exhaust gas G. A second surface 302 of the solid electrolyte element 31 is provided with the air electrode 312 exposed to air A. The exhaust electrode 311 and the air electrode 312 are disposed so as to overlap each other in the lamination direction D via the solid electrolyte element 31 at a portion on the tip side L1, which is exposed by the exhaust gas G, of the sensor element 2 in the axial direction L. A portion on the tip side L1 of the sensor element 2 in the axial direction L is provided with the detector 21 including the exhaust electrode 311 and the air electrode 312, and a portion of the solid electrolyte element 31 between the electrodes 311 and 312. The first insulator 33A is laminated on the first surface 301 of the solid electrolyte element 31. The second insulator 33B is laminated on the second surface 302 of the solid electrolyte element 31.

The solid electrolyte element 31 includes a zirconia-based oxide. The solid electrolyte element 31 includes stabilized zirconia or partially-stabilized zirconia whose main component (50 percentage by mass or more) is zirconia and part of the zirconia is displaced by a rare-earth metal element or an alkaline-earth metal element. Part of the zirconia constituting the solid electrolyte element 3 can be displaced by yttria, scandia, or calcia.

The exhaust electrode 311 and the air electrode 312 contains platinum, which is a noble metal indicating catalytic activity for oxygen, and a zirconia-based oxide serving as a material common to the solid electrolyte element 31. The common material is used for maintaining bonding strength between the solid electrolyte element 31, and the exhaust electrode 311 and the air electrode 312 formed of electrode material, when electrode material paste is printed on (applied to) the solid electrolyte element 31, and the solid electrolyte element 31 and the electrode material are baked.

As shown in FIG. 5, the exhaust electrode 311 and the air electrode 312 are connected with an electrode lead 313 for electrically connecting the electrodes 311 and 312 to an element provided outside the gas sensor 1. The electrode lead 313 is drawn out to a part on the base side L2 of the sensor element 2 in the axial direction L.

### (Gas Chamber 35)

As shown in FIG. 5 and FIG. 6, the gas chamber 35 surrounded by the first insulator 33A and the solid electrolyte element 31 are formed on and adjacent to the first surface 301 of the solid electrolyte element 31. The gas chamber 35 is formed at a position, at which the exhaust electrode 311 is accommodated, in a part on the tip side L1 of the first insulator 33A in the axial direction L. The gas chamber 35 is formed as a space closed by the first insulator 33A, a diffusion resistance portion 32, and the solid electrolyte element 31. The exhaust gas G flowing through the exhaust pipe 7 is introduced into the gas chamber 35 through the diffusion resistance portion 32.

### (Diffusion Resistance Portion 32)

As shown in FIG. 5, the diffusion resistance portion 32 of the present embodiment is adjacent to the tip side L1 of the gas chamber 35 in the axial direction L. The diffusion resistance portion 32 is disposed in an inlet which is opening and adjacent to the tip side L1 of the gas chamber 35 in the axial direction L. The diffusion resistance portion 32 is formed of porous metallic oxide such as alumina. A diffusion rate (flow rate) of the exhaust gas G introduced into the gas chamber 35 is determined by limiting a rate of the exhaust gas G passing through pores of the diffusion resistance portion 32.

The diffusion resistance portion 32 may be adjacent to the both sides of the gas chamber 35 in the width direction W. In this case, the diffusion resistance portion 32 is disposed in an inlet which is opening and adjacent to the both sides of the gas chamber 35 in the width direction W. The diffusion resistance portion 32 may be formed by using, instead of a porous body, pinholes that are small through holes communicating with the gas chamber 35.

### (Air Duct 36)

As shown in FIGS. 5 to 7, the air duct 36 surrounded by the second insulator 33B and the solid electrolyte element 31 is formed on and adjacent to the second surface 302 of the solid electrolyte element 31. The air duct 36 is formed from a part of the second insulator 33B, in which the air electrode 312 is accommodated, in the axial direction L to a base-side position exposed to the air A. At the base-side position of the sensor element 2 in the axial direction L, a base-side opening 361 serving as an air introduction portion of the air duct 36 is formed. The air duct 36 is provided from the base-side opening 361 to a position at which the air duct 36 overlaps with the gas chamber 35 in the lamination direction D via the solid electrolyte element 31. The air A is introduced into the air duct 36 from the base-side opening 361.

### (Heating Element 34)

As shown in FIGS. 5 to 7, the heating element 34 is buried in the second insulator 33B forming the air duct 36, and has a heating portion 341 generating heat by energization and a heating element lead 342 connected to the heating portion 341. The heating portion 341 is located at a position at which at least part thereof overlaps with the exhaust electrode 311 and the air electrode 312 in the lamination direction D of the solid electrolyte element 31 and the insulators 33A, 33B.

The heating element 34 has the heating portion 341 generating heat by energization and a pair of heating element leads 342 connected to the base side L2 of the heating portion 341 in the axial direction L. The heating portion 341 includes a linear conductor part having a straight part and a meandering curved part. The straight part of the heating portion 341 of the present embodiment is parallel to the axial direction L. The heating element lead 342 includes a linear conductor part parallel to the axial direction L. The resistance value of the heating portion 341 per unit length is greater than the resistance value of the heating element leads 342 per unit length. The heating element lead 342 is drawn out from the heating portion 341 to a part on the base side L2 in the axial direction L. The heating element 34 contains metallic material having conductivity.

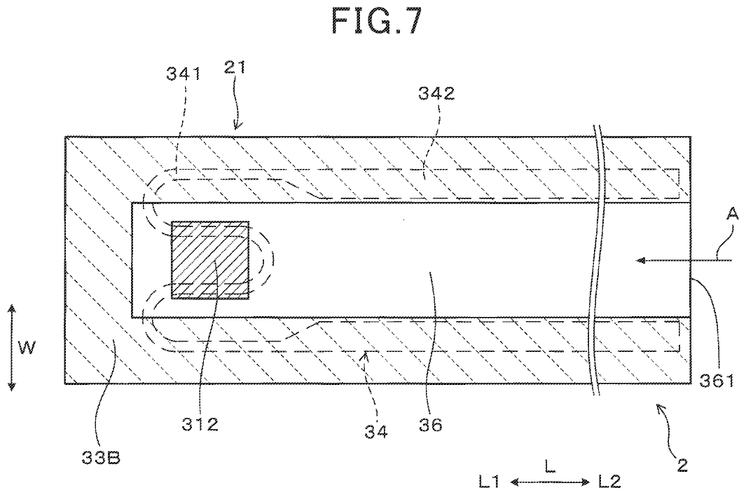

As shown in FIG. 5 and FIG. 7, the heating portion 341 of the present embodiment has a shape meandering in the axial direction L at a position on the tip side L1 of the heating element 34 in the axial direction L. The heating portion 341 may be meandering in the width direction W. The heating portion 341 is disposed at a position opposed to the exhaust electrode 311 and the air electrode 312 in the lamination direction D orthogonal to the axial direction L. In other words, in a part on the tip side L1 of the sensor element 2 in the axial direction L, the heating portion 341 is disposed at a position overlapping with the exhaust electrode 311 and the air electrode 312 in the lamination direction D.

The cross-sectional area of the heating portion 341 is smaller than the cross-sectional area of the heating element lead 342. The resistance value of the heating portion 341 per unit length is greater than the resistance value of the heating element leads 342 per unit length. The cross-sectional area herein is a cross-sectional area of a plane orthogonal to the direction in which the heating portion 341 and the heating element lead 342 extend. When voltage is applied to the pair of heating element leads 342, the heating portion 341 generates heat by Joule heat, thereby heating the periphery of the detector 21 to a target temperature.

### (Insulators 33A, 33B)

As shown in FIG. 5 and FIG. 6, the first insulator 33A forms the gas chamber 35, and the second insulator 33B forms the air duct 36. The heating element 34 is buried in the second insulator 33B. The first insulator 33A and the second insulator 33B are formed of metallic oxide such as alumina (aluminium oxide). The insulators 33A, 33B are forms as dense elements through which a gas, which is the exhaust gas G or the air A, cannot pass. The insulators 33A, 33B are provided with almost no pores through which the gas can pass.

### (Terminal Portion 22 of Sensor Element 2)

The terminal portion 22 of the sensor element 2 is electrically connected to base-side portions, in the axial direction L, of the electrode leads 313 of the exhaust electrode 311 and the air electrode 312 and the pair of heating element leads 342. The terminal portion 22 is disposed on both side surfaces of a base-side portion of the sensor element 2 in the axial direction L. The base-side portions of the electrode leads 313 and the heating element leads 342 in the axial direction L are connected to the terminal portion 22 via through holes formed in the insulators 33A, 33B.

### (Porous Layer 37)

As shown in FIG. 1, a porous layer 37 for capturing materials that would poison the exhaust electrode 311, condensed water generated in the exhaust pipe 7, and the like are provided to the entire circumference of a part on the tip side L1 of the sensor element 2 in the axial direction L. The porous layer 37 is formed of porous ceramics (metallic oxide) such as alumina. The porosity of the porous layer 37 is higher than the porosity of the diffusion resistance portion 32. The flow rate of the exhaust gas G that can pass through the porous layer 37 is higher than the flow rate of the exhaust gas G that can pass through the diffusion resistance portion 32.

As shown in FIG. 1 and FIG. 2, in the gas sensor 1 of the present embodiment, the direction orthogonal to the axial direction L of the sensor element 2 and radially extending from a central axis line of the sensor element 2 is referred to as a radial direction R. The central axis line is an imaginary line passing through the centroid of the cross section of the gas sensor 2 orthogonal to the axial direction L.

### (Housing 41)

As shown in FIG. 1, the housing 41 is used to tighten the gas sensor 1 in the opening 71 of the exhaust pipe 7. The housing 41 has a flange 411 configuring a maximum outside diameter portion, a tip-side cylinder portion 412 formed on the tip side L1 of the flange 411 in the axial direction L, and the circular protrusion 5 serving as a base-end cylinder portion formed on the base side L2 of the flange 411 in the axial direction L. The maximum outside diameter portion indicates a part whose radius dimension in the radial direction R is the largest in the housing 41. The outer periphery of the flange 411 has a hexagonal shape that is useful when the gas sensor 1 is tighten in the opening 71 for mounting by a tool. The tip-side cylinder portion 412 and the circular protrusion 5 have a cylindrical shape.

The outer periphery of a part on the base side L2 of the tip-side cylinder portion 412 in the axial direction L is provided with a male thread tightened to a female thread of the opening 71 for mounting. A portion on the tip side L1 of the tip-side cylinder portion 412 in the axial direction L is provided with an attachment portion 413 to which tip-side covers 45A, 45B described later are attached.

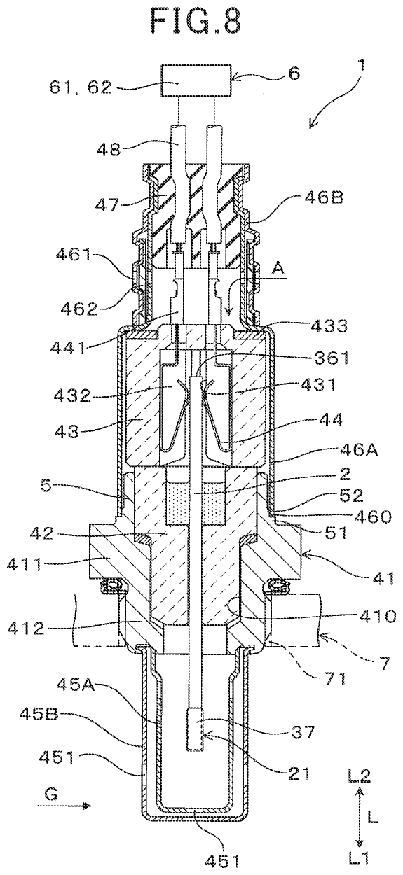

The circular protrusion 5 is provided with a caulking portion 53 for fixing a first insulator 42 to the housing 41. The caulking portion 53 is bent to the inner periphery side in the radial direction R to prevent the first insulator 42 from detaching from a central hole 410 of the housing 41 to the base side L2 in the axial direction L. As shown in FIG. 8, the first insulator 42 and the second insulator 43 may have a structure in which the circular protrusion 5 is not provided with the caulking portion 53. The caulking portion 53 may fix a cuplike sensor element 2 to the housing 41.

### (Base-Side Covers 46A, 46B)

As shown in FIG. 1, the base-side covers 46A, 46B cover a wiring part located on the base side L2 of the gas sensor 1 in the axial direction L to protect the wiring part from water and the like in the air A. The wiring part includes the contact terminal 44, which serves as a part electrically connected to the sensor element 2, and a connecting part (fitting 441) between contact terminal 44 and the lead wire 48.

The base-side covers 46A, 46B include two separate components to hold therebetween a water-repellent filter 462 for preventing water in the air A from entering the gas sensor 1. Specifically, the base-side covers 46A, 46B of the present embodiment have a first base-side cover 46A attached to the outer periphery of the circular protrusion 5 of the housing 41 and a second base-side cover 46B attached to the outer periphery on the base side L2 of the first base-side cover 46A in the axial direction L. A portion on the tip side L1 of the second base-side cover 46B in the axial direction L is attached to the outer periphery on the base side L2 of the first base-side cover 46A in the axial direction L.

An end portion on the tip side L1 of the first base-side cover 46A in the axial direction L is joined to the outer periphery of circular protrusion 5 of the housing 41 by welding or the like. Instead of the welding, the circular protrusion 5 of the housing 41 may be pressed into the end portion on the tip side L1 of the first base-side cover 46A in the axial direction L.

The sealing member 47 holding a plurality of lead wires 48 are held on the inner periphery side of a part on the base side L2 of the second base-side cover 46B in the axial direction L. The water-repellent filter 462 is held between the first base-side cover 46A and the second base-side cover 46, and between the second base-side cover 46 and the sealing member 47.

As shown in FIG. 1, a part on the tip side L1 of the second base-side cover 46B in the axial direction L is caulked to a portion on the base side L2 of the first base-side cover 46A in the axial direction L by a concave portion 463 recessed from the outer periphery side to the inner periphery side. A middle portion of the second base-side cover 46B in the axial direction L is caulked to a portion on the base side L2 of the first base-side cover 46A in the axial direction L by a concave portion 464 recessed from the outer periphery side to the inner periphery side in a state of holding the water-repellent filter 462. A portion on the base side L2 of the second base-side cover 46 in the axial direction L is caulked to the sealing member 47 by a concave portion 465 recessed from the outer periphery side to the inner periphery side in a state of holding the water-repellent filter 462.

The base-side covers 46A, 46B are disposed outside the exhaust pipe 7 of the internal-combustion engine. The gas sensor 1 of the present embodiment is for vehicles. A vehicle body to which the exhaust pipe 7 is disposed is connected to an engine compartment in which the internal-combustion engine is disposed. The air A in the engine compartment flows around the base-side covers 46A, 46B.

The second base-side cover 46B is provided with an air introduction hole 461 for introducing the air A form the outside of the gas sensor 1. The water-repellent filter 462 is disposed in a state of covering the air introduction hole 461 from the inner periphery side of the second base-side cover 46B. The base-side opening 361 of the air duct 36 of the sensor element 2 is open to space in the base-side covers 46A, 46B. The air A present around the air introduction hole 461 of the second base-side cover 46B is introduced into the base-side covers 46A, 46B through the water-repellent filter 462. The air A that has passed through the water-repellent filter 462 flows from the base-side opening 361 of the air duct 36 of the sensor element 2 into the air duct 36, and then is introduced to the air electrode 312 in the air duct 36.

### (Relationship Between Circular Protrusion 5 and Base-Side Covers 46A, 46B)

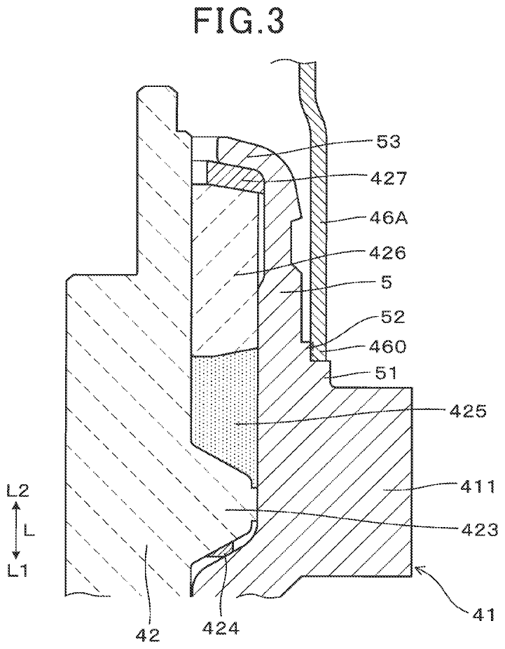

As shown in FIG. 3, the caulking portion 53 of the circular protrusion 5 protrudes from the diameter reduction portion 52 of the circular protrusion 5 to the base side L2 in the axial direction L. The outer diameter of the caulking portion 53 is smaller than the outer diameter of the diameter reduction portion 52. A circular step is formed between the root portion 51 of the circular protrusion 5 and the diameter reduction portion 52 of the circular protrusion 5. A circular step is formed also between the diameter reduction portion 52 and the caulking portion 53.

The length of the diameter reduction portion 52 in the axial direction L is short to make an area, in which the circular protrusion 5 contacts base-side covers 46A, 46B, smaller. The length of the diameter reduction portion 52 in the axial direction L is shorter than the length of the root portion 51 in the axial direction L and the length of the caulking portion 53 in the axial direction L.

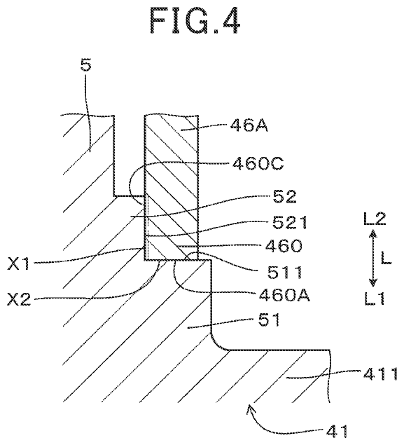

Since the end portion 460 on the tip side L1 of the base-side cover 46A in the axial direction L is attached to the outer periphery of the diameter reduction portion 52, an area in which the circular protrusion 5 contacts the base-side cover 46A can be small. As shown in FIG. 4, a joint area X1 in which the circular protrusion 5 is joined to the end portion 460 on the tip side L1 of the base-side cover 46A is larger than a contact area X2 in which the circular protrusion 5 (housing 41) contacts an end face 460A on the tip side L1 of the base-side cover 46A via a boundary face. The joint area X1 does not include the contact area X2. In FIG. 4, the joint area X1 is indicated by a chain double-dashed line. Portions at which the circular protrusion 5 contacts the base-side cover 46A are almost joined to each other by welding or the like.

More specifically, as shown in FIG. 4, the whole portions of an outer periphery 521 of the diameter reduction portion 52 of the circular protrusion 5 and an inner periphery 460C of the end portion 460 on the tip side L1 of the base-side cover 46A, which contact each other, are joined to each other by welding or the like. Although the end face 460A on the tip side L1 of the base-side cover 46A and an end face 511 on the base side L2 of the root portion 51 of the circular protrusion 5 contact each other, they are not joined to each other. An area X1 in which the outer periphery 521 of the diameter reduction portion 52 of the circular protrusion 5 is joined to the inner periphery 460C of the end portion 460 on the tip side L1 of the base-side cover 46A is larger than an area X2 in which the end face 460A on the tip side L1 of the base-side cover 46A contacts the end face 511 on the base side L2 of the root portion 51 of the circular protrusion 5. Since the joint area X1 is larger than the contact area X2, heat transfer from the circular protrusion 5 to the base-side cover 46A can be effectively suppressed while maintaining joint strength between the circular protrusion 5 and the base-side cover 46A.

When the base-side cover 46A is joined to the housing 41, the end portion 460 on the tip side L1 of the base-side cover 46A is attached to the outer periphery 521 of the diameter reduction portion 52 of the circular protrusion 5. Then, a laser beam is radiated from the outer periphery of the end portion 460 on the tip side L1 of the base-side cover 46A to melt the diameter reduction portion 52 and the end portion 460 on the tip side L1 of the base-side cover 46A and join them to each other.

The length of portions of the outer periphery 521 of the diameter reduction portion 52 of the circular protrusion 5 and the inner periphery 460C of the end portion 460 on the tip side L1 of the base-side cover 46A joined to each other in the axial direction L may be in a range of 1 to 3 mm. Hence, since the length of the portions of the circular protrusion 5 and the base-side covers 46A, 46B joined to each other is short, the temperature of the sealing member 47 heated by heat transferred from the base-side covers 46A, 46B can be lowered.

### (First Insulator 42)

As shown in FIG. 1, the first insulator 42 is disposed in the central hole 410 penetrating a central portion of the housing 41 in the axial direction L. The first insulator 42 is formed of an insulating ceramic material. A first insertion hole 420 penetrating in the axial direction L is formed in a central portion of the first insulator 42 to insert the sensor element 2 thereinto. An end portion on the base side L2 of the first insertion hole 420 in the axial direction L is provided with a concave portion 421 for fixing in which glass powder 422 for fixing the sensor element 2 is disposed. The sensor element 2 is fixed to the first insulator 42 by the glass powder 422 disposed in the concave portion 421 in a state where the sensor element 2 is inserted into the first insertion hole 420 of the first insulator 42.

The outer periphery of the first insulator 42 is provided with a protrusion 423 forming a maximum outer diameter portion of the first insulator 42. In a state where the first insulator 42 is disposed in the central hole 410 of the housing 41, a sealing material 424 is disposed on the tip side L1 of the protrusion 423 in the axial direction L in the central hole 410, and caulking materials 425, 426, 427 are disposed on the base side L2 of the protrusion 423 in the axial direction L. The caulking materials include a powder sealing material 425, a cylindrical body 426, and a caulking material 427. The caulking portion 53 of the circular protrusion 5 of the housing 41 is bent to the inner periphery side in the radial direction R to fix the first insulator 42 in the central hole 410 of the housing 41 by caulking via the sealing material 424 and the caulking materials 425, 426, 427.

### (Second Insulator 43)

As shown in FIG. 1, the second insulator 43 is disposed on the base side L2 of the first insulator 42 in the axial direction L to hold the contact terminal 44 contacting the terminal portion 22 of the sensor element 2. The second insulator 43 is formed of an insulating ceramic material. A second insertion hole 431 penetrating in the axial direction L is formed in a central portion of the second insulator 43 to insert the sensor element 2 thereinto. A groove 432 in which the contact terminal 44 is disposed is formed at a position of the second insulator 43 communicating with the second insertion hole 431. The second insulator 43 is disposed on the inner periphery side of the base-side cover 46A in the radial direction R. The second insulator 43 is pressed against the first insulator 42 via a plate spring 433 by the first base-side cover 46A.

### (Contact Terminal 44)

As shown in FIG. 1, the contact terminal 44 contacts the terminal portion 22 of the sensor element 2 to electrically connect the terminal portion 22 to the lead wire 48. The contact terminal 44 is disposed in the groove 432 of the second insulator 43. The contact terminal 44 is connected to the lead wire 48 via the fitting 441 and contacts the terminal portion 22 by the action of restoring force of elastic deformation. A plurality of contact terminals 44 are disposed, the number of the contact terminals 44 corresponding to the number of the terminal portions 22 of the sensor element 2, in other words, corresponding the number of the electrode leads 313 of the exhaust electrode 311 and the air electrode 312, and the pair of heating element leads 342.

### (Sealing Member 47 and Lead Wire 48)

As shown in FIG. 1, the sealing member (bush) 47 is disposed on the inner periphery side of the second base-side cover 46B to hold the plurality of lead wires 48 by sealing. Since the sealing member 47 functions as a sealing material, the sealing member 47 is formed of a rubber material having elastic deformability. The sealing member 47 is provided with a through-hole into which the lead wire 48 is inserted. The second base-side cover 46B is caulked to the sealing member 47, whereby gaps between the lead wires 48 and the through-holes and a gap between the sealing member 47 and the second base-side cover 46B are sealed. The lead wire 48 connects the contact terminals 44 to a sensor control unit 6 provided outside the gas sensor 1. The lead wire 48 is formed by covering an inner conductor by a covering layer.

### (Tip-Side Covers 45A, 45B)

As shown in FIG. 1, the tip-side covers 45A, 45B cover the detector 21 of the sensor element 2 protruding from the end face on the tip side L1 of the housing 41 in the axial direction L to the tip side L1. The tip-side covers 45A, 45B are attached to the outer periphery of the attachment portion 413 provided to the tip-side cylinder portion 412 of the housing 41. The tip-side covers 45A, 45B of the present embodiment has a double structure of the first tip-side cover 45A and the second tip-side cover 45B covering the first tip-side cover 45A. The first tip-side cover 45A and the second tip-side cover 45B are provided with gas flow holes 451 through which the exhaust gas G can flow.

The detector 21 of the sensor element 2 and the tip-side covers 45A, 45B are disposed in the exhaust pipe 7 of the internal-combustion engine. Part of the exhaust gas G flowing through the exhaust pipe 7 flows from the gas flow holes 451 of the tip-side covers 45A, 45B into the tip-side covers 45A, 45B. The exhaust gas G in the tip-side covers 45A, 45B passes through the porous layer 37 and the diffusion resistance portion 32 of the sensor element 2 and is introduced to the exhaust electrode 311. The tip-side covers 45A, 45B may have a single structure in which the gas flow holes 451 is formed.

### (Sensor Control Unit 6)

As shown in FIG. 1, the lead wire 48 of the gas sensor 1 is electrically connected to the sensor control unit 6 that controls gas detection of the gas sensor 1. The sensor control unit 6 performs electrical control of the gas sensor 1 in cooperation with an engine control unit that controls combustion driving of the engine. As shown in FIG. 5, the sensor control unit 6 includes a current measurement circuit 61 that measures a current flowing between the exhaust electrode 311 and the air electrode 312, a voltage application circuit 62 that applied voltage between the exhaust electrode 311 and the air electrode 312, an energizing circuit for energizing the heating element 34, and the like. The sensor control unit 6 may be configured in the engine control unit.

### (Another Gas Sensor 1)

The gas sensor 1 may detect a concentration of a specified gas component such as NOx (nitrogen oxide). In the NOx sensor, a pump electrode that pumps oxygen to the air electrode 312 by application of voltage is disposed on the upstream side of the flow of exhaust gas G contacting the exhaust electrode 311 in the solid electrolyte element 31. The air electrode 312 is also provided at a position at which the air electrode 312 overlaps with the pump electrode in the lamination direction D via the solid electrolyte element 31.

The gas sensor 1 may include, instead of the lamination type sensor element 2, a cuplike sensor element 2 including the solid electrolyte element 31 having a bottomed cylindrical shape. In the cuplike sensor element 2, the exhaust electrode 311 is provided on the outer periphery of the solid electrolyte element 31, and the air electrode 312 is provided on the inner periphery of the solid electrolyte element 31. The cuplike sensor element 2 is disposed in the central hole 410 of the housing 41 without using the first insulator 4. Also in this case, the base-side cover 46A can be attached to the outer periphery of the circular protrusion 5 of the housing 41.

### (Effects)

In the gas sensor 1 of the present embodiment, an area of a part where the housing 41 and the base-side cover 46A contact each other is set as small as possible so that heat transfer from the housing 41 to the base-side covers 46A, 46B is difficult to occur. Specifically, according to the structure in which the end portion 460 of the base-side cover 46A on the tip side L1 in the axial direction L is jointed to the outer periphery of the diameter reduction portion 52 of the circular protrusion 5 provided to the housing 41, an area of a portion where the outer periphery of the housing 41 and the inner periphery of the base-side cover 46A contact each other can be limited to an area of a portion where the outer periphery 521 of the diameter reduction portion 52 of the circular protrusion 5 is joined to the inner periphery 460C of the end portion 460 of the base-side cover 46A on the tip side L1 in the axial direction L. Thereby, the area of the part where the housing 41 and the base-side cover 46A contact each other can be as small as possible. Hence, heat is difficult to transfer from the housing 41 to the base-side cover 46A, and heat is difficult to transfer from the base-side covers 46A, 46B to the sealing member 47 disposed on the inner periphery side of the second base-side cover 46B.

Therefore, according to the gas sensor 1 of the present embodiment, it is difficult to heat the sealing member 47 by heat transferred from the housing 41, whereby the sealing member 47 can be effectively protected from the heat.

### Second Embodiment

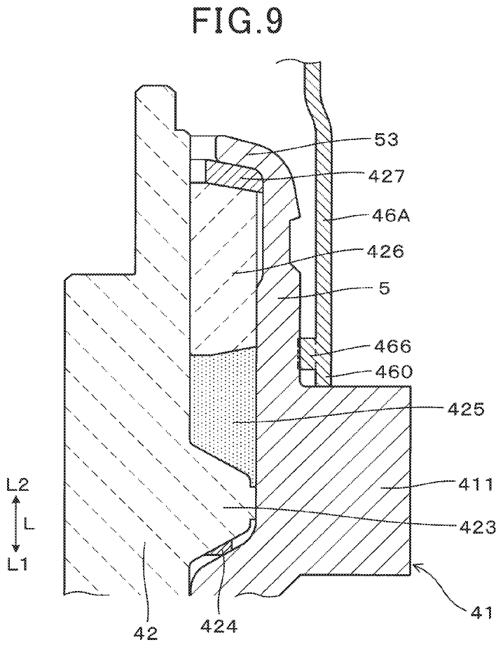

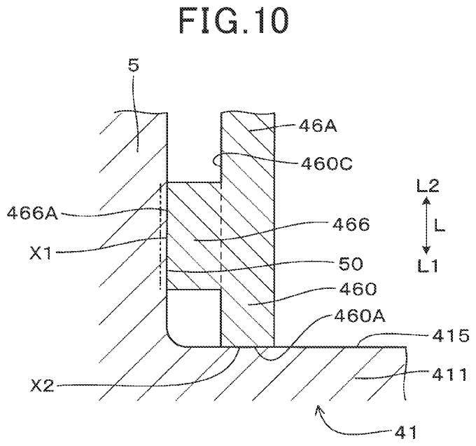

In the present embodiment, a structure of a portion where the housing 41 and the base-side cover 46A contact each other is different from the structure of the first embodiment. As shown in FIG. 9 and FIG. 10, the inner periphery 460C of the end portion 460 on the tip side L1 of the base-side cover 46A in the axial direction L of the present embodiment is provided with a convex portion 466 joined to an outer periphery 50 of the circular protrusion 5 of the housing 41. The convex portion 466 is circularly formed on the entire circumference of a part of the inner periphery 460C of the end portion 460 on the tip side L1 of the base-side cover 46A in the axial direction L. The convex portion 466 has a shape in which a cross section orthogonal to the axial direction L has a constant shape in the axial direction L. The circular protrusion 5 and the end portion 460 on the tip side L1 of the base-side cover 46A is jointed to each other via the convex portion 466.

The convex portion 466 has a short length in the axial direction L so that an area in which the circular protrusion 5 contacts the base-side cover 46A is small. The length of the convex portion 466 in the axial direction L is shorter than the length of the circular protrusion 5 in the axial direction L.

As shown in FIG. 9, a part on the base side L2 of the circular protrusion 5 in the axial direction L is provided with the caulking portion 53 for fixing a first insulator 42 to the housing 41. The circular protrusion 5 protrudes to the outer periphery side from a root portion on the tip side L1 of the caulking portion 53 in the axial direction L. The first insulator 42 and the second insulator 43 may have a structure in which the circular protrusion 5 is not provided with the caulking portion 53.

Since the convex portion 466 of the base-side cover 46A is attached to the outer periphery of the circular protrusion 5, an area in which the circular protrusion 5 contacts the base-side cover 46A can be small. As shown in FIG. 10, a joint area X1 in which the circular protrusion 5 is joined to the convex portion 466 of the end portion 460 on the tip side L1 of the base-side cover 46A is larger than a contact area X2 in which the housing 41 contacts the end face 460A on the tip side L1 of the base-side cover 46A via a boundary face.

More specifically, as shown in FIG. 10, the whole portions of the outer periphery 50 of the circular protrusion 5 and an inner periphery 466A of the convex portion 466, which contact each other, are joined to each other by welding or the like. Although the end face 460A on the tip side L1 of the base-side cover 46A and a surface 415 of the housing 41 contact each other, they are not joined to each other. An area X1 in which the outer periphery 50 of the circular protrusion 5 is joined to the inner periphery 466A of the convex portion 466 is larger than an area X2 in which the end face 460A on the tip side L1 of the base-side cover 46A contacts the surface 415 of the housing 41.

The length of portions of the outer periphery 50 of the circular protrusion 5 and the inner periphery 466A of the convex portion 466 of the base-side cover 46A joined to each other in the axial direction L may be in a range of 1 to 3 mm. Hence, since the length of the portions of the circular protrusion 5 and the base-side cover 46A joined to each other is short, the temperature of the sealing member 47 heated by heat transferred from the base-side covers 46A, 46B can be lowered.

In the present embodiment, according to the structure in which the convex portion 466 provided to the inner periphery 460C of the base-side cover 46A is jointed to the outer periphery 50 of the circular protrusion 5, an area of a part where the outer periphery of the housing 41 and the inner periphery of the base-side cover 46A contact each other can be limited to an area of a part where the circular protrusion 5 is joined to the convex portion 466. Hence, heat is difficult to transfer from the housing 41 to the base-side covers 46A, 46B and the sealing member 47.

Other configurations and effects of the gas sensor 1 of the present embodiment are similar to those of the first embodiment. In the present embodiment, components denoted by the same reference numerals as those indicated in the first embodiment are similar components to those in the first embodiment.

### Third Embodiment

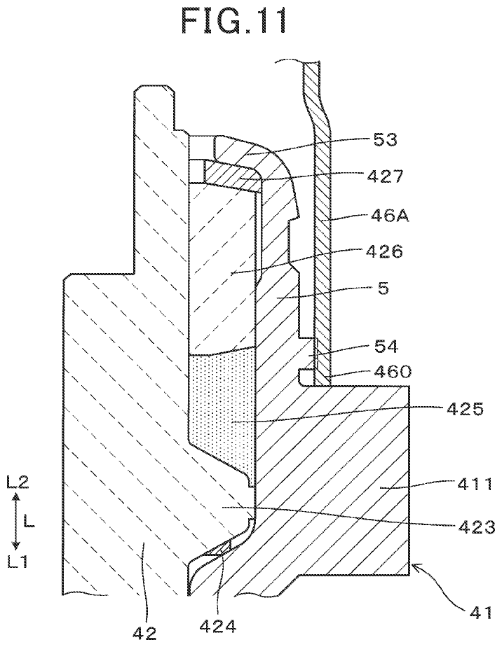

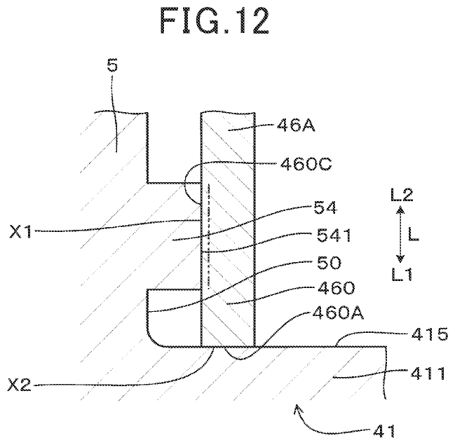

In the present embodiment, the convex portion 54 in the second embodiment is provided to the circular protrusion 5. As shown in FIG. 11 and FIG. 12, the outer periphery 50 of the circular protrusion 5 of the housing 41 in the present embodiment is provided with the convex portion 54 joined to the inner periphery 460C of the end portion 460 on the tip side L1 of the base-side cover 46A in the axial direction L. The convex portion 54 is circularly formed on the entire circumference of a part of the outer periphery 50 of the circular protrusion 5 in the axial direction L. The convex portion 54 has a shape in which a cross section orthogonal to the axial direction L has a shape constant in the axial direction L. The circular protrusion 5 and the end portion 460 on the tip side L1 of the base-side cover 46A is jointed to each other via the convex portion 54.

The convex portion 54 may be formed integrally with the outer periphery 50 of the circular protrusion 5 of the housing 41 when the housing 41 is formed. The convex portion 54 may be formed by joining a cylindrical member to the outer periphery 50 of circular protrusion 5 of the housing 41 by welding or the like.

The length of portions of the outer periphery 541 of the convex portion 54 of the circular protrusion 5 and the inner periphery 460C of the base-side cover 46A joined to each other in the axial direction L may be in a range of 1 to 3 mm. Hence, since the length of the portions of the circular protrusion 5 and the base-side cover 46A joined to each other is short, the temperature of the sealing member 47 heated by heat transferred from the base-side covers 46A, 46B can be lowered.

In the present embodiment, as shown in FIG. 4, a joint area X1 in which the convex portion 54 of the circular protrusion 5 is joined to the end portion 460 on the tip side L1 of the base-side cover 46A is larger than a contact area X2 in which the housing 41 contacts the end face 460A on the tip side L1 of the base-side cover 46A via a boundary face. More specifically, the whole portions of the outer periphery 541 of the convex portion 54 of the circular protrusion 5 and the inner periphery 460C of the end portion 460 on the tip side L1 of the base-side cover 46A, which contact each other, are joined to each other by welding or the like. Although the end face 460A on the tip side L1 of the base-side cover 46A and the surface 415 of the housing 41 contact each other, they are not joined to each other. An area X1 in which the outer periphery 541 of the convex portion 54 of the circular protrusion 5 is joined to the inner periphery 460C of the end portion 460 on the tip side L1 of the base-side cover 46A is larger than an area X2 in which the end face 460A on the tip side L1 of the base-side covers 46A, 46B contacts the surface 415 of the housing 41.

Other configurations and effects of the gas sensor 1 of the present embodiment are similar to those of the first and second embodiments. In the present embodiment, components denoted by the same reference numerals as those indicated in the first and second embodiments are similar components to those in the first and second embodiment.

### Forth Embodiment

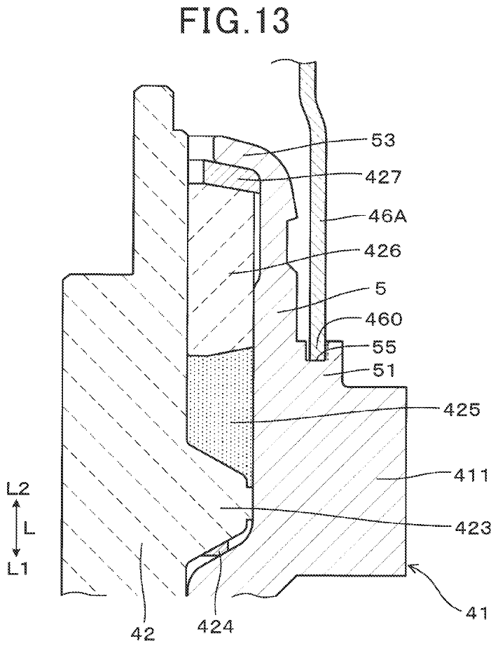

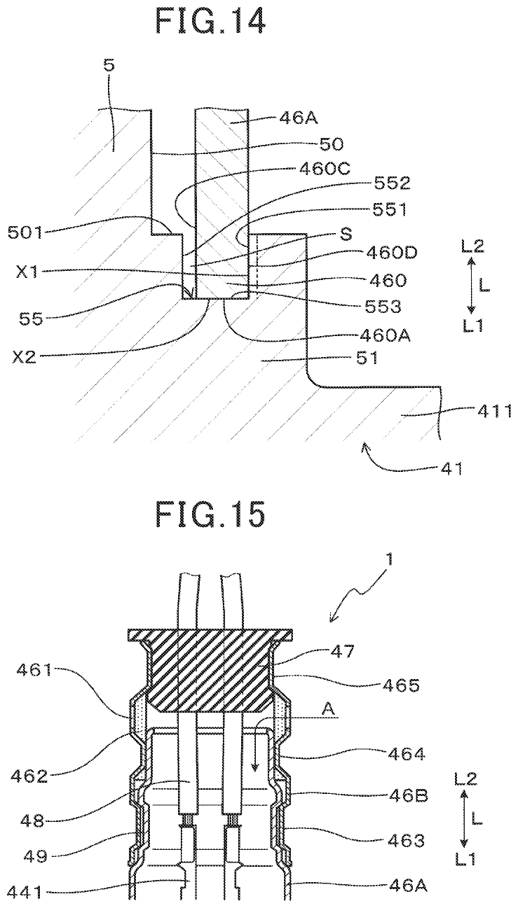

In the present embodiment, a structure of a part where the housing 41 and the base-side cover 46A contact each other is different from the structures of the first to third embodiments. As shown in FIG. 13 and FIG. 14, an end face 501 on the base side L2 of the root portion 51 in the axial direction L, which is connected to the flange 411, of the circular protrusion 5 of the housing 41 of present embodiment is provided with a concave portion 55 in which the end portion 460 on the tip side L1 of the base-side cover 46A in the axial direction L is disposed. The concave portion 55 is circularly formed on the entire circumference of a part of the end face 501 on the base side L2 of the root portion 51 of the circular protrusion 5 in the radial direction R. The end portion 460 on the tip side L1 of the base-side cover 46A is jointed to the root portion 51 in a state in which the end portion 460 is disposed in the concave portion 55.

An outer periphery 460D of the end portion 460 on the tip side L1 of the base-side cover 46A is joined to an inner wall surface 551 of the concave portion 55 on the outer periphery side. A gap S is provided between the inner periphery 460C of the end portion 460 on the tip side L1 of the base-side cover 46A and an inner wall surface 552 of the concave portion 55 on the inner periphery side.

The length of the concave portion 55 in the axial direction L is short to make an area, in which the circular protrusion 5 contacts base-side cover 46A, smaller. The depth of the concave portion 55 in the axial direction L is shorter than the length of the circular protrusion 5 in the axial direction L.

As shown in FIG. 13, a part on the base side L2 of the circular protrusion 5 in the axial direction L is provided with the caulking portion 53 for fixing a first insulator 42 to the housing 41. The root portion 51 of the circular protrusion 5 protrudes to the outer periphery side from a root portion on the tip side L1 of the caulking portion 53 in the axial direction L. The first insulator 42 and the second insulator 43 may have a structure in which the circular protrusion 5 is not provided with the caulking portion 53.

Since the end portion 460 on the tip side L1 of the base-side cover 46A is attached to the concave portion 55 of the circular protrusion 5, an area in which the circular protrusion 5 contacts the base-side cover 46A can be small. As shown in FIG. 14, a joint area X1 in which the concave portion 55 of the circular protrusion 5 is joined to the end portion 460 on the tip side L1 of the base-side cover 46A is larger than a contact area X2 in which the concave portion 55 of the circular protrusion 5 contacts the end face 460A on the tip side L1 of the base-side covers 46A, 46B via a boundary face.

More specifically, as shown in FIG. 14, the whole portions of the inner wall surface 551 on the outer periphery side of the concave portion 55 of the circular protrusion 5 and the outer periphery 460D of the end portion 460 on the tip side L1 of the base-side cover 46A, which contact each other, are joined to each other by welding or the like. Although a bottom face 553 on the tip side L1 of the concave portion 55 of the circular protrusion 5 and the end face 460A on the tip side L1 of the base-side cover 46A contact each other, they are not joined to each other. An area X1 in which the inner wall surface 551 on the outer periphery side of the concave portion 55 of the circular protrusion 5 is joined to the outer periphery 460D of the end portion 460 on the tip side L1 of the base-side cover 46A is larger than an area X2 in which the bottom face 553 on the tip side L1 of the concave portion 55 of the circular protrusion 5 contacts the end face 460A on the tip side L1 of the base-side cover 46A.

The length of portions of the inner wall surface 551 on the outer periphery side of the concave portion 55 of the circular protrusion 5 and the outer periphery 460D of the end portion 460 of the base-side cover 46A joined to each other in the axial direction L may be in a range of 1 to 3 mm. Hence, since the length of the portions of the circular protrusion 5 and the base-side cover 46A joined to each other is short, the temperature of the sealing member 47 heated by heat transferred from the base-side covers 46A, 46B can be lowered.

In the present embodiment, according to the structure in which the end portion 460 on the tip side L1 of the base-side cover 46A contacts the concave portion 55 of the root portion 51 of the circular protrusion 5, an area of a part where the housing 41 and the base-side cover 46A contact each other can be limited to an area of a part where the concave portion 55 contacts the end portion 460 on the tip side L1 of the base-side cover 46A. Hence, heat is difficult to transfer from the housing 41 to the base-side covers 46A, 46B and the sealing member 47.

Other configurations and effects of the gas sensor 1 of the present embodiment are similar to those of the first to third embodiments. In the present embodiment, components denoted by the same reference numerals as those indicated in the first to third embodiments are similar components to those in the first to third embodiments.

Although not shown, in the gas sensor 1 of the present embodiment, the inner periphery 460C of the end portion 460 on the tip side L1 of the base-side cover 46A may be joined to the inner wall surface 552 on the inner periphery side of the concave portion 55. In this case, the gap S is provided between the outer periphery 460D of the end portion 460 on the tip side L1 of the base-side cover 46A and the inner wall surface 551 on the outer periphery side of the concave portion 55.

### Fifth Embodiment

The gas sensor 1 of the present embodiment is devised to suppress heat transfer through the base-side covers 46A, 46B. Specifically, as shown in FIG. 15, a heat insulator 49, whose heat conductivity is lower than the heat conductivity of the first base-side cover 46A and the heat conductivity of the second base-side cover 46B, is held between the first base-side cover 46A and the second base-side cover 46B.

The first base-side cover 46A and the second base-side cover 46B are formed of metallic material. The heat insulator 49 of the present embodiment is formed of glass wool made of sheet-shaped glass fiber. The heat insulator 49 may be formed of various materials that can suppress heat transfer between the first base-side cover 46A and the second base-side cover 46B. For example, the heat insulator 49 may be formed of porous ceramic material in which gaps having complex shapes, regularly arranged through-holes, or the like are formed.

The heat insulator 49 is held at a position on the tip side L1 in the axial direction L with respect to the position at which a water-repellent filter 462 is held between the first base-side cover 46A and the second base-side cover 46B. The concave portion 463 caving from the outer periphery side to the inner periphery side is formed at a position at which the heat insulator 49 is held between the first base-side cover 46A and the second base-side cover 46B.

According to the gas sensor 1 of the present embodiment, the heat insulator 49 can suppress heat transfer from the first base-side cover 46A to the second base-side cover 46B. Hence, heat transfer from the tip side L1 to the base side L2 of the base-side covers 46A, 46B in the axial direction L can be suppressed, whereby the sealing member 47 can be effectively protected from heat.

Other configurations and effects of the gas sensor 1 of the present embodiment are similar to those of the first to fourth embodiments. In the present embodiment, components denoted by the same reference numerals as those indicated in the first to fourth embodiments are similar components to those in the first to fourth embodiments.

### <Confirmation Test>

In the present confirmation test, for the gas sensor 1 of the first embodiment in which the length of portions of the diameter reduction portion 52 of the circular protrusion 5 and the base-side cover 46A, which are joined to each other, in the axial direction L is in a range of 1 to 3 mm, a simulation was performed for degrees of heating in parts of the gas sensor 1 when the exhaust gas G whose temperature is 1100° C. is flowing through the exhaust pipe 7 in which the gas sensor 1 is disposed. For comparison, a similar simulation was performed for a gas sensor of a comparison embodiment in which the length of portions of the circular protrusion 5 and the base-side cover 46A, which are joined to each other, in the axial direction L is 4 mm. Also in this simulation, temperature distributions of the parts of the gas sensor 1 are displayed. Then, temperatures of the sealing members 47 of the gas sensor 1 of the first embodiment and the gas sensor of the comparison embodiment were confirmed.

The temperature of the sealing member 47 of the gas sensor of the comparison embodiment was approximately 260° C. The temperature of the sealing member 47 of the gas sensor 1 of the first embodiment was approximately 235° C. when the length of the joined portions is 1 mm, approximately 243° C. when the length of the joined portions is 2 mm, and approximately 254° C. when the length of the joined portions is 3 mm. It was found that, in the case of the first embodiment, compared with the case of the comparison embodiment, the temperature of the sealing member 47 can be lowered by approximately 6 to 25° C., whereby the sealing member 47 can be effectively protected from heat.

In addition, the degree of lowering the temperature of the sealing member 47 was confirmed by a simulation for a case in which the heat insulator 49 of the fifth embodiment is applied to the gas sensor 1 of the first embodiment. The simulation was performed for a case in which glass wool is used for the heat insulator 49 and a case in which rustless steel mesh is used for the heat insulator 49.

The heat conductivity of glass wool is 0.05 [W/m*K], and the heat conductivity of rustless steel mesh is 16 [W/m*K]. The heat conductivity of rustless steel forming the first base-side cover 46A and the second base-side cover 46B is 16 [W/m*K].

It is found that, in the gas sensor 1 to which the heat insulator 49 formed of glass wool is applied, compared with the gas sensor 1 of the first embodiment, the temperature of the sealing member 47 can be lowered by approximately 18° C. In addition, in the gas sensor 1 to which the heat insulator 49 formed of rustless steel mesh is applied, compared with the gas sensor 1 of the first embodiment, the temperature of the sealing member 47 can be lowered by approximately 8° C.

The present disclosure is not limited to the embodiments, and can constitute further different embodiments within a range that does not deviate from the gist thereof. The present disclosure includes various modifications and modifications within an equivalent range. Combinations of various components assumed from the present disclosure are also included in the technical idea of the present disclosure.

The present disclosure provides a gas sensor that makes it difficult to heat a sealing member by heat transferred from a housing, whereby the sealing member can be effectively protected from the heat.

As a first aspect of the present disclosure, a gas sensor (1) is provided which includes:

a sensor element (2) having a detector (21) capable of detecting gas and located on a tip side in an axial direction (L);

a cylindrical housing (41) into which the sensor element is inserted in a state in which the detector protrudes to a tip side (L1) in the axial direction;

a contact terminal (44) that contacts a terminal portion (22) provided on a base side position of the sensor element in the axial direction;

an insulator (43) that holds the contact terminal;

a lead wire (48) that is connected to the contact terminal and is externally drawn out;

a sealing member (47) that holds the lead wire; and

a base-side cover (46A, 46B) that is attached to an outer periphery of a circular protrusion (5) provided to protrude from a flange (411), which configures a maximum outer diameter portion of the housing, to a base side (L2) in the axial direction, and holds the sealing member on an inner periphery side.

The circular protrusion is provided with a root portion (51) that protrudes to the base side of the flange in the axial direction, a diameter reduction portion (52) which is located on the base side of the root portion in the axial direction and whose outer periphery has a diameter reduced with respect to that of the root portion to form a circular step between the diameter reduction portion and the root portion, and a general portion which is located on the base side of the diameter reduction portion in the axial direction and whose outer periphery has a diameter reduced with respect to that of the diameter reduction portion to form a circular step between the general portion and the diameter reduction portion.

A tip-side end portion (460) of the base-side cover in the axial direction is jointed to the outer periphery of the diameter reduction portion.

A circular gap whose width in a radial direction orthogonal to the axial direction is smaller than a thickness of the base-side cover is provided between the base-side cover and the general portion.

As a second aspect of the present disclosure, a gas sensor (1) is provided which includes:

a sensor element (2) having a detector (21) capable of detecting gas and located on a tip side in an axial direction (L);

a cylindrical housing (41) into which the sensor element is inserted in a state in which the detector protrudes to a tip side (L1) in the axial direction;

a contact terminal (44) that contacts a terminal portion (22) provided on a base side position of the sensor element in the axial direction;

an insulator (43) that holds the contact terminal;

a lead wire (48) that is connected to the contact terminal and is externally drawn out;

a sealing member (47) that holds the lead wire; and

a base-side cover (46A, 46B) that is attached to an outer periphery of a circular protrusion (5) provided to protrude from a flange (411), which configures a maximum outer diameter portion of the housing, to a base side (L2) in the axial direction, and holds the sealing member on an inner periphery side.

An inner periphery of a tip-side end portion (460) of the base-side cover in the axial direction is provided with a convex portion (466) in a state where an outer periphery of the tip-side end portion is parallel to the axial direction,

The circular protrusion and the tip-side end portion of the base-side cover is joined to each other via the convex portion.

A circular gap whose width is the same as a width of the convex portion in a radial direction orthogonal to the axial direction is provided between the base-side cover and the circular protrusion.

As a third aspect of the present disclosure, a gas sensor (1) is provided which includes:

a sensor element (2) having a detector (21) capable of detecting gas and located on a tip side in an axial direction (L);

a cylindrical housing (41) into which the sensor element is inserted in a state in which the detector protrudes to a tip side (L1) in the axial direction;

a contact terminal (44) that contacts a terminal portion (22) provided on a base side position of the sensor element in the axial direction;

an insulator (43) that holds the contact terminal;

a lead wire (48) that is connected to the contact terminal and is externally drawn out;

a sealing member (47) that holds the lead wire; and

a base-side cover (46A, 46B) that is attached to an outer periphery of a circular protrusion (5) provided to protrude from a flange (411), which configures a maximum outer diameter portion of the housing, to a base side (L2) in the axial direction, and holds the sealing member on an inner periphery side.

an end face (501) on the base side of the circular protrusion in the axial direction is provided with a concave portion (55).

a tip-side end portion (460) of the base-side cover is joined to the circular protrusion in a state in which the tip-side end portion is disposed in the concave portion.

### (Gas Sensor of the First Aspect)

In the gas sensor of the first aspect, an area of a part where the housing and the base-side cover contact each other is set as small as possible so that heat transfer from the housing to the base-side cover is difficult to occur. Specifically, the circular protrusion provided to the housing has a root portion connected to the flange and the diameter reduction portion whose outer periphery has a diameter reduced with respect to that of the root portion. The tip-side end portion of the base-side cover in the axial direction is jointed to the outer periphery of the diameter reduction portion of the circular protrusion.

According to the configuration, the length of the diameter reduction portion of the circular protrusion in the axial direction can be short, so that the area in which the tip-side end portion of the base-side cover in the axial direction contacts the diameter reduction portion of the circular protrusion can be small. Thereby, the area of the part where the housing and the base-side cover contact each other can be as small as possible. Hence, heat is difficult to transfer from the housing to the base-side cover, and heat is difficult to transfer from the base-side cover to the sealing member disposed on the inner periphery side of the base-side cover.

Therefore, according to the gas sensor of the first aspect, it is difficult to heat the sealing member by heat transferred from the housing, whereby the sealing member can be effectively protected from the heat.

### (Gas Sensor of the Second Aspect)

Also in the gas sensor of the second aspect, an area of a part where the housing and the base-side cover contact each other is set as small as possible so that heat transfer from the housing to the base-side cover is difficult to occur. Specifically, at least one of the outer periphery of the circular protrusion provided to the housing and the inner periphery of the tip-side end portion of the base-side cover in the axial direction is provided with the convex portion. The circular protrusion and the tip-side end portion of the base-side cover are joined to each other via the convex portion.

According to the configuration, the length of the convex portion in the axial direction can be short, so that the area in which the convex portion contacts the base-side cover or the circular protrusion can be small. Thereby, the area of the part where the housing and the base-side cover contact each other can be as small as possible. Hence, heat is difficult to transfer from the housing to the base-side cover, and heat is difficult to transfer from the base-side cover to the sealing member disposed on the inner periphery side of the base-side cover.

Therefore, also according to the gas sensor of the second aspect, it is difficult to heat the sealing member by heat transferred from the housing, whereby the sealing member can be effectively protected from the heat.

### (Gas Sensor of the Third Aspect)

Also in the gas sensor of the third aspect, an area of a part where the housing and the base-side cover contact each other is set as small as possible so that heat transfer from the housing to the base-side cover is difficult to occur. Specifically, the end face on the base side of the root portion, which is connected to the flange, in the axial direction is provided with the concave portion. The tip-side end portion of the base-side cover is joined to the circular protrusion in a state in which the tip-side end portion is disposed in the concave portion.

According to the configuration, the length of the concave portion in the axial direction can be short, so that the area in which the concave portion contacts the base-side cover can be small. Thereby, the area of the part where the housing and the base-side cover contact each other can be as small as possible. Hence, heat is difficult to transfer from the housing to the base-side cover, and heat is difficult to transfer from the base-side cover to the sealing member disposed on the inner periphery side of the base-side cover.

Therefore, also according to the gas sensor of the third aspect, it is difficult to heat the sealing member by heat transferred from the housing, whereby the sealing member can be effectively protected from the heat.

What is claimed is:

1. A gas sensor, comprising:
a sensor element having a detector capable of detecting gas and located on a tip side in an axial direction;
a cylindrical housing into which the sensor element is inserted in a state in which the detector protrudes to a tip side in the axial direction;
a contact terminal that contacts a terminal portion provided on a base side position of the sensor element in the axial direction;
an insulator that holds the contact terminal;
a lead wire that is connected to the contact terminal and is externally drawn out;
a sealing member that holds the lead wire; and
a base-side cover that is attached to an outer periphery of a circular protrusion provided to protrude from a flange, which configures a maximum outer diameter portion of the housing, to a base side in the axial direction, and holds the sealing member on an inner periphery side, wherein
the circular protrusion is provided with a root portion that protrudes to the base side of the flange in the axial direction, a diameter reduction portion which is located on the base side of the root portion in the axial direction and whose outer periphery has a diameter reduced with respect to that of the root portion to form a circular step between the diameter reduction portion and the root portion, and a general portion which is located on the base side of the diameter reduction portion in the axial direction and whose outer periphery has a diameter reduced with respect to that of the diameter reduction portion to form a circular step between the general portion and the diameter reduction portion,
a tip-side end portion of the base-side cover in the axial direction is jointed to the outer periphery of the diameter reduction portion, and
a circular gap whose width in a radial direction orthogonal to the axial direction is smaller than a thickness of the base-side cover is provided between the base-side cover and the general portion.

2. The gas sensor according to claim 1, wherein
length of the diameter reduction portion in the axial direction is shorter than length of the root portion in the axial direction and length of the general portion in the axial direction.

3. A gas sensor, comprising:
a sensor element having a detector capable of detecting gas and located on a tip side in an axial direction;
a cylindrical housing into which the sensor element is inserted in a state in which the detector protrudes to a tip side in the axial direction;
a contact terminal that contacts a terminal portion provided on a base side position of the sensor element in the axial direction;
an insulator that holds the contact terminal;
a lead wire that is connected to the contact terminal and is externally drawn out;
a sealing member that holds the lead wire; and
a base-side cover that is attached to an outer periphery of a circular protrusion provided to protrude from a flange, which configures a maximum outer diameter portion of the housing, to a base side in the axial direction, and holds the sealing member on an inner periphery side, wherein
an inner periphery of a tip-side end portion of the base-side cover in the axial direction is provided with a convex portion in a state where an outer periphery of the tip-side end portion is parallel to the axial direction,
the circular protrusion and the tip-side end portion of the base-side cover is joined to each other via the convex portion, and
a circular gap whose width is the same as a width of the convex portion in a radial direction orthogonal to the axial direction is provided between the base-side cover and the circular protrusion.

4. A gas sensor, comprising:
a sensor element having a detector capable of detecting gas and located on a tip side in an axial direction;
a cylindrical housing into which the sensor element is inserted in a state in which the detector protrudes to a tip side in the axial direction;
a contact terminal that contacts a terminal portion provided on a base side position of the sensor element in the axial direction;
an insulator that holds the contact terminal;
a lead wire that is connected to the contact terminal and is externally drawn out;
a sealing member that holds the lead wire; and
a base-side cover that is attached to an outer periphery of a circular protrusion provided to protrude from a flange, which configures a maximum outer diameter portion of the housing, to a base side in the axial direction, and holds the sealing member on an inner periphery side, wherein
an end face on the base side of the circular protrusion in the axial direction is provided with a concave portion, and
a tip-side end portion of the base-side cover is joined to the circular protrusion in a state in which the tip-side end portion is disposed in the concave portion.

5. The gas sensor according to claim 1, wherein
a joint area in which the circular protrusion is joined to the tip-side end portion of the base-side cover is larger than a contact area in which the housing contacts the tip-side end portion of the base-side cover via a boundary face.

6. The gas sensor according to claim 3, wherein
a joint area in which the circular protrusion is joined to the tip-side end portion of the base-side cover is larger than a contact area in which the housing contacts the tip-side end portion of the base-side cover via a boundary face.

7. The gas sensor according to claim 4, wherein
a joint area in which the circular protrusion is joined to the tip-side end portion of the base-side cover is larger than a contact area in which the housing contacts the tip-side end portion of the base-side cover via a boundary face.

8. The gas sensor according to claim 1, wherein
the circular protrusion is provided with a caulking portion for fixing the insulator or the sensor element to the housing.

9. The gas sensor according to claim 3, wherein
the circular protrusion is provided with a caulking portion for fixing the insulator or the sensor element to the housing.

10. The gas sensor according to claim 4, wherein
the circular protrusion is provided with a caulking portion for fixing the insulator or the sensor element to the housing.

11. The gas sensor according to claim 1, wherein
the base-side cover includes a first base-side cover that is attached to an outer periphery of the circular protrusion and a second base-side cover that is attached to an outer periphery at a base-side position of the first base-side cover in the axial direction and holds the sealing member on an inner periphery side, and
a heat insulator, whose heat conductivity is lower than heat conductivity of the first base-side cover and heat conductivity of the second base-side cover, is held between the first base-side cover and the second base-side cover.

12. The gas sensor according to claim 3, wherein
the base-side cover includes a first base-side cover that is attached to an outer periphery of the circular protrusion and a second base-side cover that is attached to an outer periphery at a base-side position of the first base-side cover in the axial direction and holds the sealing member on an inner periphery side, and
a heat insulator, whose heat conductivity is lower than heat conductivity of the first base-side cover and heat conductivity of the second base-side cover, is held between the first base-side cover and the second base-side cover.

13. The gas sensor according to claim 4, wherein
the base-side cover includes a first base-side cover that is attached to an outer periphery of the circular protrusion and a second base-side cover that is attached to an outer periphery at a base-side position of the first base-side cover in the axial direction and holds the sealing member on an inner periphery side, and
a heat insulator, whose heat conductivity is lower than heat conductivity of the first base-side cover and heat conductivity of the second base-side cover, is held between the first base-side cover and the second base-side cover.
